(19) United States
(12) Patent Application Publication
(10) Pub. No.: US 2004/0063678 A1
(43) Pub. Date: Apr. 1, 2004

(54) Dexamethasone Gel

(75) Inventors: Gunther Bellmann, Berlin (DE); Gudrun Claus-Herz, Berlin (DE); Christoph Kessler, Berlin (DE)

Correspondence Address: Bausch & Lomb Incorporated, One Bausch & Lomb Place, Rochester, NY, 14604-2701, US

(73) Assignee: Bausch & Lomb Incorporated, Rochester, NY (US)

(21) Appl. No.: 10/665,937

(22) Filed: Sep. 18, 2003

Related U.S. Application Data
(63) Continuation of application No. 09/486,460, filed on May 22, 2000, now abandoned.
Application No. 09/486,460 is a 371 of international application No. PCT/EP98/06339, filed on May 10, 1998.

(30) Foreign Application Priority Data
Oct. 6, 1997 (DE) 197 44 113.0

(51) Int. Cl. A61K 31/573

(52) U.S. Cl. 514/179

(57) ABSTRACT

The invention relates to an ophthalmological preparation containing dexamethasone as the active ingredient and, optionally, also containing the usual additives and water, which is characterized by a content of at least one gel-forming pharmacologically acceptable substance in a quantity sufficient for adjusting the viscosity of the preparation as a gel.

The invention relates to an ophthalmological preparation containing dexamethasone as the active ingredient and, optionally, also containing the usual additives and water.

Dexamethasone preparations are known in the form of eye-drops and eye ointments. Eye-drops usually have concentrations between 0.05 and 0.1%, whereas eye ointments usually contain about 0.05% dexamethasone sodium phosphate.

Known eye-drop formulations comprising dexamethasone esters as the active ingredient are adjusted to slightly alkaline pH values. For example, solutions of drops which are currently available commercially typically have a pH value of about 7.3. These slightly alkaline values are selected because dexamethasone esters manifest greatest stability in the slightly alkaline range, as is also the case with prednisolone esters.

In this regard, the pH value is typically selected to be as close as possible to neutral, since higher alkalinities are suspected of causing eye irritations and other tolerance problems.

It is known that the antiphlogistic efficacy of eye-drops is superior to that of eye ointments. In experiments, the bio-availability of eye ointments was substantially poorer than that of eye-drop solutions, even in cases of extended contact times (Cox et al., Arch. Ophthalmol. 88, 549 (1972); Kupferman et al., Arch. Ophthalmol. 91, 373 (1974)).

Although eye-drops manifest a greater bio-availability than ointments, it is often difficult to achieve the desired dwell time, when using solutions of drops. Drop solutions are washed away relatively rapidly, for example by lacrimal fluid, with the result that the concentration of the active ingredient decreases relatively rapidly.

For many applications, it would be advantageous if it were possible to achieve an extended constant concentration of the active ingredient on the eye, after a once-only application. In this regard, recourse may be had to ointments only at the price of considerable drawbacks, owing to the known bio-availability problems.

It is the object of the invention to provide an ophthalmological preparation which does not involve these difficulties.

This object is met by the features as defined in the attached principal claim.

Advantageous further developments and embodiments are defined in the subordinate claims.

The attempt to formulate a gel preparation according to the invention, on the basis of the known drop solutions, such that a suitable gelling agent is added, leads to surprising difficulties.

When working on the basis of the known drop solutions in the pH range of 7 to about 7.3, the addition of carbomer surprisingly leads to a considerable decomposition of the active ingredient after only a short interval, such that the required stability is not achieved.

It appears that there is an intensified conversion of dexamethasone dihydrogen phosphate disodium (“dexamethasone sodium phosphate) into the free base.

Surprisingly, it is possible for this decomposition to be prevented by selecting a higher pH value. Gelling agents, such as Carbopol^{(R)}, appear to have an adverse effect on the durability of aqueous dexamethasone phosphate solutions in the acid to neutral range and also in the weakly alkaline range, while, surprisingly, distinctly improving said durability in the more alkaline range.

Accordingly, when carbomers, in particular-of the Carbopol 980 NF type or similar Carbopol products, are used as gelling agents in the gels according to the invention, the required stability is provided in the pH range above 7.3.

Storable stable gel preparations at pH values above 7.3, preferably above 7.6 and most preferably at about 7.8 and above, are provided at a concentration of 0.05 to about 1% by mass, preferably between 0.1 and 0.6% by mass and, most preferably, at about 3% by mass of carbomer, in particular of the Carbopol 980 NF type, and active-ingredient concentrations of the order of about 0.1% by mass. The upper limit depends on the ophthalmological compatibility of the preparation, since very high alkalinities may lead to irritations. Accordingly, pH values above 8 will not be used in practice, or only under exceptional circumstances.

The preparation according to the invention is usually adjusted to the required pH value by means of pharmacologically acceptable alkalis, for example sodium hydroxide.

In addition to the active ingredient, the gelling agent and the alkali for adjusting the alkalinity, it also contains a preservative, such as, in particular, benzododecinium chloride (BAC C12), from the group including the benzalkonium chlorides. In addition, the preparation preferably contains an isotonic agent, such as sorbitol, and a chelating agent, such as sodium edetate. Water is used as the solvent for the preparation.

A typical formulation according to the invention for a batch of 100 kg substantially contains the following components:

| Designation | Quantity | |
|---|---|---|
| Dexamethasone dihydrogen phosphate disodium | 0.0985 | kg |
| Benzododecinium chloride (BAC C12) | 0.0100 | kg |
| Carbopol 980 NF | 0.3000 | kg |
| Sorbitol | 4.9000 | kg |
| Sodium hydroxide, solid | 0.1460-0.1540 | kg |
| Sodium edetate | 0.0100 | kg |
| Water for injection purposes | 94.5275-94.5355 | kg |
| | 100.000 | kg |

Surprisingly, a preparation of this kind is as stable as a comparable solution of drops, i.e. two or three years. The preparation is very well tolerated when applied, does not cause eye irritations (with the choice of the preservative contributing in this regard), and permits the desired extended dwell time, in the course of which the bio-availability of the active ingredient is not adversely affected, in contrast to drop solutions.

1. Ophthalmological preparation containing dexamethasone as the active ingredient and, optionally, also containing the usual additives and water, characterized by a content of at least one gel-forming pharmacologically acceptable substance in a quantity sufficient for adjusting the viscosity of the preparation as a gel.

2. Preparation according to claim 1, characterized in that the preparation contains at least one carbomer, in particular of the Carbopol 980 NF type, as the gelling agent.

3. Preparation according to claim 1 or claim 2, characterized in that the preparation contains 0.05 to 1% by mass, preferably between 0.1 and 0.6% by mass and, most preferably, about 0.3% by mass of carbomer.

4. Preparation according to one of claims 1 to 3, characterized in that the preparation contains dexamethasone dihydrogen phosphate disodium in a concentration of about 0.1% by mass as the active ingredient.

5. Preparation according to one of claims 1 to 4, characterized in that the preparation has a pH value above 7.3, preferably between 7.6 and 8.2 and, most preferably, between 7.8 and 8.0.

6. Preparation according to one of claims 1 to 5, characterized in that the preparation contains benzododecinium chloride (BAC C12).
